(19) United States
(12) Patent Application Publication
(10) Pub. No.: US 2011/0178332 A1
(43) Pub. Date: Jul. 21, 2011

(54) CATALYST FOR GAS-PHASE CONTACT OXIDATION OF HYDROCARBON, PREPARATION METHOD THEREOF AND GAS-PHASE OXIDATION METHOD OF HYDROCARBON USING THE SAME

(75) Inventors: Jin-Do Kim, Daejeon (KR); Kyong-Yong Cha, Daejeon (KR); Bu-Young Jo, Seoul (KR); Sang-Heup Moon, Seoul (KR)

(73) Assignees: LG CHEM, LTD, Seoul (KR); SNU R&DB FOUNDATION, Seoul (KR)

(21) Appl. No.: 12/997,214

(22) PCT Filed: Jun. 9, 2009
(86) PCT No.: PCT/KR09/03080

§ 371 Date: Mar. 28, 2011

(30) Foreign Application Priority Data
Jun. 10, 2008 (KR) 10-2008-0054037
Aug. 11, 2008 (KR) 10-2008-0078273

(51) Int. Cl. B01J 27/057; B01J 37/08; C07C 51/16

(52) U.S. Cl. 562/549; 502/215

(57) ABSTRACT

The present invention provides a catalyst for use in gas-phase contact oxidation of hydrocarbon with an improved yield and selectivity, a preparation method thereof, and a method of a gas-phase oxidation of the hydrocarbon using the same. The catalyst comprises a composite metal oxide of Mo, V, Te and Nb; and a tungsten or tungsten oxide attached to the composite metal oxide, wherein an atomic molar ratio of the tungsten attached to the composite metal oxide to the molybdenum contained in the composite metal oxide ranges from 0.00001:1 to 0.02:1.

### FIELD OF THE INVENTION

The present invention relates to a catalyst for use in gas-phase contact oxidation of hydrocarbon, a preparation method thereof, and a method of a gas-phase oxidation of the hydrocarbon using the same. More specifically, the present invention relates to a catalyst for use in gas-phase contact oxidation of hydrocarbon with an improved yield and selectivity, a preparation method thereof, and a method of a gas-phase oxidation of the hydrocarbon using the same.

### BACKGROUND OF THE INVENTION

There are continuous attempt to change from propylene or isobutylene to cheap hydrocarbon such as propane or isobutane as a starting material for producing acrylic acid, methacrylic acid or acrylonitrile.

The composite metal oxide catalyst, for example MoVTeNbO-based catalyst has been developed for oxidation of hydrocarbon such as propane or isobutane to produce acrylic acid, methacrylic acid or acrylonitrile. However, the composite metal oxide catalyst has a low conversion rate of hydrocarbon, and for example, a low selectivity to conversion of the hydrocarbon to acrylic acid, and etc. The composite metal oxide catalyst cannot provide the production of acrylic acid, and etc with sufficiently high yield and selectivity.

Therefore, there is still need for a catalyst with more improved catalytic activity and selectivity, but is a limit to the improvement in higher activity and selectivity of the catalyst.

There were some attempts to develop the catalyst with improved selectivity and activity by adding other metal to the composite metal oxide catalyst.

For example, U.S. Pat. No. 5,380,933 discloses a catalyst including a composite metal oxide of Mo—V—Te with addition of Nb, Ta, W, Ti, Al, Zr, Cr or Mn. In addition, EP 0 767 164 B1, U.S. Pat. No. 6,036,880, U.S. Pat. No. 5,231,214, U.S. Pat. No. 5,281,745 or U.S. Pat. No. 5,472,925 disclose a catalyst including a composite metal oxide of Mo—V—Sb (or Te) with addition of Ti, Al, W, Ta, Sn, Fe, Co or Ni.

However, in these catalysts, for example, a MoVTeNbO-based composite metal oxide as a main component and added component cannot be bound efficiently and the added component cannot be contained in a preferred ratio. Therefore, there is a limit to the improvement in reaction yield and selectivity for oxidizing the hydrocarbon such as propane or isobutane in gaseous phase. So far, there is no catalyst having yield and selectivity which is enough for being used in commercially available level.

### SUMMARY OF THE INVENTION

The present invention provides a catalyst for use in gas-phase contact oxidation reaction of hydrocarbon such as propane or isobutane where the catalyst has an improved yield and selectivity to the oxidation reaction.

Also, the present invention provides a method of preparing catalyst for the gas-phase contact oxidation of the hydrocarbon.

The present invention also provides a method of the gas-phase contact oxidation for the hydrocarbon by using the catalyst at high yield and selectivity.

### DETAILED DESCRIPTION OF THE EMBODIMENTS

The present invention provides a catalyst for gas-phase contact oxidation of a hydrocarbon, comprising a composite metal oxide of Molybdenum (Mo), Vanadium (V), Tellurium (Te) and Niobium (Nb); and a tungsten (W) or tungsten oxide attached to the composite metal oxide, wherein an atomic molar ratio of the tungsten attached to the composite metal oxide to the molybdenum contained in the composite metal oxide ranges from 0.00001:1 to 0.02:1, more preferably 0.0001:1 to 0.01:1, or most preferably 0.0001:1 to 0.002:1.

The catalyst comprises a composite metal oxide of Molybdenum (Mo), Vanadium (V), Tellurium (Te) and Niobium (Nb) represented by chemical formula I; and a tungsten (W) or tungsten oxide attached to the composite metal oxide:

Mo_{1.0}VₐTe_{b}Nb_{c}Oₙ (I)

where,

a, b, or c is independently an atomic molar ratio of Vanadium, Tellurium, or Niobium, provided that 0.01≦a≦1, and preferably 0.2≦a≦0.4, 0.01≦b≦1 and preferably 0.1≦b≦0.3, and 0.01≦c≦1 and preferably 0.05≦c≦0.2; and

n is an atomic molar ratio of Oxygen that is determined by valence and atomic molar ratio of Vanadium, Tellurium, and Niobium.

The present invention provides a method of preparing a catalyst for gas-phase contact oxidation of hydrocarbon according to claim 1, comprising the steps of: preparing a first mixture of Molybdenum (Mo) precursor, Vanadium (V) precursor, Tellurium (Te) precursor, Niobium (Nb) precursor, and acid; preparing a composite metal oxide of Molybdenum (Mo), Vanadium (V), Tellurium (Te) and Niobium (Nb) by calcining the first mixture; preparing a second mixture of the composite metal oxide and tungsten precursor; and calcining the second mixture.

The present invention provides a method of a gas-phase oxidation of hydrocarbon, comprising oxidizing the hydrocarbon in the presence of the catalyst in gaseous phase.

The gas-phase oxidation of the hydrocarbon containing propane, isobutane, or etc. can produce for examples, acrylic acid, methacrylic acid or acrylonitrile with high yield and selectivity.

Hereinafter, the catalyst for use in gas-phase contact oxidation of hydrocarbon, a preparation method thereof, and a method of a gas-phase oxidation of the hydrocarbon using the same are described in more detail according to specific embodiments of the present invention.

In an embodiment, a catalyst for gas-phase contact oxidation of a hydrocarbon, comprising a composite metal oxide of Molybdenum (Mo), Vanadium (V), Tellurium (Te) and Niobium (Nb); and a tungsten (W) or tungsten oxide attached to the composite metal oxide, wherein an atomic molar ratio of the tungsten to molybdenum contained in the composite metal oxide ranges from 0.00001:1 to 0.02:1.

In the catalyst for use in gas-phase contact oxidation of hydrocarbon, the term, “attachment” of the tungsten or tungsten oxide to the composite metal oxide means that the tungsten or tungsten oxide does not form the chemical bond with each component of the composite metal oxide such as Molybdenum (Mo), Vanadium (V), Tellurium (Te) and Niobium (Nb), but merely is adhered via non-chemical or physical force, for examples, an attractive force between the metal atoms or an attractive force between the metal atom and the oxygen atom. Hereinafter, otherwise the term is defined specifically, the terms “attachment”, “attached” and “attaching” are used as defined above.

In addition, the term, “gas-phase contact oxidation” or “gas-phase oxidation” means any reaction that aliphatic hydrocarbon, and preferably alkane including propane, isobutane or etc. is oxidized in gaseous phase to produce unsaturated carboxylic acid or unsaturated nitrile such as acrylic acid, methacrylic acid or acrylonitrile.

For example, the term “gas-phase contact oxidation” or “gas-phase oxidation” can be defined to compass the broad meanings including a “direct oxidation” that the aliphatic hydrocarbon is oxidized to produce unsaturated carboxylic acid, and an “ammoxidation” that the aliphatic hydrocarbon is oxidized to produce unsaturated nitrile. Hereinafter, otherwise the term is defined specifically; the terms “gas-phase contact oxidation” or “gas-phase oxidation” are used as defined above.

In the catalyst according to the embodiment, tungsten (W) or tungsten oxide is attached via non-chemical binding to the surface of the composite metal oxide of Molybdenum (Mo), Vanadium (V), Tellurium (Te) and Niobium (Nb). Particularly, an atomic molar ratio of attached the tungsten to the molybdenum contained in the composite metal oxide to ranges from 0.00001:1 to 0.02:1.

As a result of study, the present inventors found that tungsten (W) or tungsten oxide is attached via non-chemical or physical binding to the surface of the composite metal oxide and can act as another catalytic site being distinct from the composite metal oxide itself.

In particular, the tungsten or tungsten oxide is attached to the composite metal oxide so as to satisfy a specific atomic molar ratio of the tungsten attached to the composite metal oxide to the molybdenum contained in the composite metal oxide (i.e., 0.00001:1 to 0.02:1), thereby making tungsten or tungsten oxide act most effectively as a different catalytic site with maintaining catalytic activity of the composite metal oxide. Accordingly, the catalyst for use in gas-phase contact oxidation of hydrocarbon shows more excellent catalytic activity and selectivity.

The catalyst of the embodiment shows excessively excellent catalytic activity and selectivity compared to the MoVTeNbO-based composite metal oxide alone. More surprisingly, the catalyst has still more excellent catalytic activity and selectivity than the five-membered composite metal oxide where the tungsten is chemically bound to MoVTeNbO-based composite metal oxide, and than a catalyst having an atomic molar ratio of the tungsten attached to the composite metal oxide to the molybdenum contained in the composite metal oxide beyond the ranges of 0.00001:1 to 0.02:1.

This is because tungsten chemically bound to the MoVTeNbO-based composite metal oxide has a difficult in acting as another catalytic site. Also, in case that the atomic molar ratio of tungsten to the molybdenum is beyond the ranges of 0.00001:1 to 0.02:1, especially, over 0.02:1, the tungsten attached to the composite metal oxide can inhibit the catalytic site of the composite metal oxide itself. On the other hand, in the catalyst of the embodiment, the tungsten or tungsten oxide can act effectively as another catalytic site with maintaining an excellent catalytic activity of the composite metal oxide itself, because tungsten or tungsten oxide attaches via non-chemical or physical binding to the surface of the MoVTeNbO-based composite metal oxide at a specific range of atomic molar ratio of the tungsten to molybdenum.

Therefore, the catalyst for use in gas-phase contact oxidation of hydrocarbon according to the embodiment can selectively oxidizing the hydrocarbon such as propane or isobutane to produce acrylic acid, methacrylic acid or acrylonitrile at high yield and selectivity.

Meanwhile, in the catalyst of the embodiment, the composite metal oxide may be a composite metal oxide of Molybdenum (Mo), Vanadium (V), Tellurium (Te) and Niobium (Nb) represented by chemical formula I. The tungsten or tungsten oxide can be attached to the composite metal oxide.

Mo_{1.0}VₐTe_{b}Nb_{c}Oₙ (I)

where,

a, b, or c is independently an atomic molar ratio of Vanadium, Tellurium, or Niobium, provided that 0.01≦a≦1, and preferably 0.2≦a≦0.4, 0.01≦b≦1 and preferably 0.1≦b≦0.3, and 0.01≦c≦1 and preferably 0.05≦c≦0.2; and

n is an atomic molar ratio of Oxygen that is determined by valence and atomic molar ratio of Vanadium, Tellurium, and Niobium.

Molybdenum, Vanadium, Tellurium and Niobium bind chemically at the specific atomic molar ratio to form the composite metal oxide, thereby making the composite metal oxide itself be more excellent activity, and easily forming the composite metal oxide.

Furthermore, in the catalyst of the embodiment, the tungsten or tungsten oxide is attached to the composite metal oxide so that the atomic molar ratio of the tungsten to the molybdenum contained in the composite metal oxide ranges from 0.00001:1 to 0.02:1, more preferably 0.0001:1 to 0.01:1, or most preferably 0.0001:1 to 0.002:1.

As described above, as the tungsten or tungsten oxide is attached to the composite metal oxide at the specific range, the catalyst shows an excellent catalytic activity and selectivity. However, when the attached molar ratio of the tungsten or tungsten oxide to the molybdenum is excessively lower than 0.00001:1, the catalyst shows an unimproved catalytic activity and selectivity, which is similar to those of the composite metal oxide alone without being attached with the tungsten or tungsten oxide. In addition, if the attached molar ratio of the tungsten or tungsten oxide is excessively higher than 0.02:1, the additional improvement in the catalytic activity and selectivity cannot be achieved, and rather, the tungsten or tungsten oxide can inhibit and deteriorate the activity of composite metal oxide itself. In particular, if the catalyst comprises the tungsten or tungsten oxide at an atomic molar ratio of tungsten to molybdenum more than 0.02:1, it shows the catalytic activity and selectivity similar to the composite metal oxide alone. Thus, the catalyst of the embodiment can show more excellent catalytic activity and selectivity, when the attached molar ratio of the tungsten to molybdenum ranges from 0.00001:1 to 0.02:1, more preferably 0.0001:1 to 0.01:1, and most preferably 0.0001:1 to 0.002:1.

Because the catalyst for use in gas-phase contact oxidation of hydrocarbon has more excellent catalytic activity and selectivity, it can be preferably applied for gas-phase oxidation of hydrocarbon such as propane, isobutane, and etc.

Particularly, the catalyst can be used effectively for selective producing acrylic acid, methacrylic acid or acrylonitrile from propane or isobutane at high yield and selectivity.

In another embodiment of the invention, a method of preparing a catalyst for gas-phase contact oxidation of hydrocarbon is provided.

The method of preparing the catalyst comprises the steps of: preparing a first mixture of Molybdenum (Mo) precursor, Vanadium (V) precursor, Tellurium (Te) precursor, Niobium (Nb) precursor, and acid; preparing a composite metal oxide of Molybdenum (Mo), Vanadium (V), Tellurium (Te) and Niobium (Nb) by calcining the first mixture; preparing a second mixture of the composite metal oxide and tungsten precursor; and calcining the second mixture.

In the method, after the composite metal oxide is formed from Molybdenum (Mo) precursor, Vanadium (V) precursor, Tellurium (Te) precursor, and Niobium (Nb) precursor, it is mixed and calcined with a tungsten precursor to produce the catalyst. As a result, the catalyst comprising the composite metal oxide and the tungsten or tungsten oxide attached to the composite metal oxide via non-chemical or physical binding.

By using the specific amount of the tungsten precursor, the embodiment provides the catalyst including the tungsten (W) or tungsten oxide which is attached at a specific atomic molar ratio of the tungsten to molybdenum contained in the composite metal oxide ranging from 0.00001:1 to 0.02:1. The amount of tungsten precursor to satisfy the atomic molar ratio can be easily determined by a person of an ordinary skill in the art in consideration of amounts of other precursors and reaction condition.

In the method of preparing the catalyst, Molybdenum (Mo) precursor, Vanadium (V) precursor, Tellurium (Te) precursor and Niobium (Nb) precursor can be selected from metal precursors which have been used for preparing the composite metal oxide without any limitation.

For example, the molybdenum precursor includes ammonium molybdate, ammonium paramolybdate, ammonium heptamolybdate, molybdenum oxide (MoO₃ or MoO₂), molybdenum chloride (MoCl₅ or MoCl₄), molybdenum acetylacetonate, Phosphomolybdic acid and silicomolybdic acid, and etc., and more preferably ammonium molybdate, ammonium paramolybdate, and ammonium heptamolybdate. The examples of the vanadium precursor include ammonium metavanadate, vanadium oxide (V₂O₅ or V₂O₃), vanadium chloride (VCl₄), vanadium, vanadyl acetylacetonate, and etc., and more preferably ammonium metavanadate. The examples of Tellurium precursor include telluric acid, tellurium oxide (TeO₂), tellurium chloride (TeCl₄), telluric acetylacetonate, and etc., and more preferably telluric acid. The examples of Niobium precursor include niobium hydrogen oxalate, ammonium niobium oxalate, niobium oxide (Nb₂O₅), niobium chloride (NbCl₅), niobic acid, niobium tartarate, and etc., and more preferably ammonium niobium oxalate.

Besides the examples of metal precursors, any Molybdenum (Mo) precursor, Vanadium (V) precursor, Tellurium (Te) precursor and Niobium (Nb) precursor which have been used formerly can be used for preparing the composite metal oxide catalyst without any limitation.

In an embodiment of the method, the acid mixed with the precursors of the molybdenum, vanadium, tellurium and niobium can adjust pH of the first mixture suitably, thereby effective forming the composite metal oxide of the molybdenum, vanadium, tellurium and niobium. The acid can be any inorganic acid, for examples, at least one selected from the group consisting of nitric acid, sulfuric acid, hydrochloric acid, carbonic acid, hypochlorous acid and fluoric acid.

In the method of the embodiment, the acid is mixed with the precursors of molybdenum, vanadium, tellurium and niobium to prepare the first mixture that may be aqueous solution made by dissolving the components in aqueous solvent such as water. The composite metal oxide can be prepared from the first mixture in aqueous solution according to general hydrothermal reaction.

The composite metal oxide is produced by calcining the first mixture. For example, when the first mixture is in aqueous solution, the first mixture may be dried and pulverized to make a particle with a certain diameter, and then be calcined.

In the process of the composite metal oxide, the dry step, for example, can be carried out at 100-150□ for enough time for complete drying of the first mixture. The pulverizing step, for example, can be performed by the dried first mixture to be a particle with diameter of 100-300 μm, and more preferably 180-250 μm. To obtain the particle, the first mixture can be pulverized and formed to compressed powder, and then be pulverized. The calcining step, for example, can be carried out at 200-700° C. for 1 to 10 hours in the atmosphere of air or nitrogen, or under the vacuum. More specifically, the calcining step can be performed at 200-400° C. for 1 to 5 hours in the atmosphere of air, and then re-performed at 500-700° C. for 1 to 5 hours in the nitrogen atmosphere.

After forming the composite metal oxide of molybdenum, vanadium, tellurium and niobium, the second mixture is prepared by mixing and calcining the composite metal oxide and tungsten precursor to obtain a catalyst for use in gas-phase contact oxidation of hydrocarbon

The tungsten precursor can be any tungsten precursor which has been used formerly for making the catalyst including tungsten without any limitation. The examples of tungsten precursor include ammonium meta tungstate, tungstenic acid, ammonium tungsten pentahydrate or tetraamine tungsten nitrate, but not limited thereto.

Like the first mixture, the second mixture can be in aqueous solution, which can be dried and calcined to produce the catalyst for use in gas-phase contact oxidation of hydrocarbon. The drying step can be performed at 50-150° C. for 0.5 to 5 hours. The calcining step can be carried out at 300-700□ for 1 to 5 hours in the nitrogen atmosphere.

According to the preparation method as described above, the catalyst according to the embodiment of the invention can be obtained where the tungsten is attached to the composite metal oxide at a specific atomic molar ratio.

Because the catalyst shows improved catalytic activity and selectivity, it can be properly used for gas-phase oxidation of hydrocarbon including propane, isobutane or etc. to selectively produce acrylic acid, methacrylic acid, acrylonitrile, and etc.

In another embodiment, a method of a gas-phase oxidation of hydrocarbon, comprising oxidizing the hydrocarbon in the presence of the catalyst in gaseous phase is provided.

In the gas-phase oxidation, the use of catalyst having an improved catalytic activity and selectivity makes the selective preparation of acrylic acid, methacrylic acid or acrylonitrile with high yield from hydrocarbon including propane or isobutane.

The method of the gas-phase oxidation of hydrocarbon can be performed according the general method considering the kind of the reactant (i.e., the hydrocarbon) and the product.

For example, when propane or isobutane is direct oxidized according to the gas-phase oxidation to obtain acrylic acid or methacrylic acid, the gas-phase oxidation reaction can be performed at 200-600□ in the oxygen and nitrogen atmosphere. The gas-phase oxidation can be performed by feeding propane, oxygen and nitrogen at volumetric speed of 500-3000 hr⁻¹ to reactor and the reactor can be fixed bed type reactor used widely.

On the other hand, when acrylonitrile is produced by gas-phase oxidation of propane, according to general reaction condition, gas-phase ammoxidation of propane can be performed at 300-600° C. in the oxygen and nitrogen atmosphere.

### EXAMPLES

A better understanding of the invention may be obtained in light of the following examples to illustrate, but are not to be construed to limit, the invention.

### Comparative Example 1

At room temperature, 0.232 g of ammonium metavanadate, 0.349 g of telluric acid and 1.178 g of ammonium paramolybdate were dissolved in 500 of distilled water to produce a solution.

The solution was added by 0.238 g of ammonium niobium oxalate dissolved in 4 ml, of distilled water and then agitated for 180 minutes to a produce a mixture solution. The mixture solution was added by 0.04 g of nitric acid and agitated for 60 minutes.

Then, the distilled water was evaporated with rotary depression dryer and dried completely at 120° C. The dried product was pulverized to make a compressed powder, pulverized again, and selected to obtain particle with diameter of about 180 to 250 μm. The selected particles was calcined at 200° C. for 2 hours in the air, and then calcined secondly at 600° C. for 2 hours in the nitrogen atmosphere. As a result, the composite metal oxide, Mo_{1.0}V_{0.3}Te_{0.23}Nb_{0.12}Oₙ was produced.

### Example 1

2 g of the composite metal oxide of Comparative Example 1 was mixed with 50 g of distilled water, added by 0.00034 g of ammonium tungsten pentahydrate solution (10%), and then agitated for 180 minutes. After agitation, the resultant was dried at 80° C. for 60 minutes, and re-dried in oven at 120° C. for 480 minutes. The dried product was calcined at 300° C. for 2 hours in the nitrogen atmosphere.

As a result, the catalyst of Example 1 including the tungsten or tungsten oxide attached to the composite metal oxide (Mo_{1.0}V_{0.3}Te_{0.23}Nb_{0.12}Oₙ) at an atomic molar ratio of Mo to W of 1:0.000013 was obtained.

### Example 2

2 g of the composite metal oxide of Comparative Example 1 was mixed with 50 g of distilled water, added by 0.00056 g of ammonium tungsten pentahydrate solution (10%), and then agitated for 180 minutes. After agitation, the resultant was dried at 80° C. for 60 minutes, and re-dried in oven at 120° C. for 480 minutes. The dried product was calcined at 300° C. for 2 hours in the nitrogen atmosphere.

As a result, the catalyst of Example 2 including the tungsten or tungsten oxide attached to the composite metal oxide (Mo_{1.0}V_{0.3}Te_{0.23}Nb_{0.12}Oₙ) at an atomic molar ratio of Mo to W of 1:0.000022 was obtained.

### Examples 3 to 9

The catalysts were prepared according to the substantially same method of Examples 1 and 2, except that the added amount of ammonium tungsten pentahydrate solution was different to achieve an atomic molar ratio of Mo to W as described in Table 1. The catalysts of Examples 3 to 9 were obtained to include the tungsten or tungsten oxide attached to the composite metal oxide, Mo_{1.0}V_{0.3}Te_{0.23}Nb_{0.12}Oₙ.

TABLE 1

| | Atomic molar ratio |
|---|---|
| No. of Example | of Mo to W (Mo:W) |
| Example 1 | 1:0.000013 |
| Example 2 | 1:0.000022 |
| Example 3 | 1:0.00013 |
| Example 4 | 1:0.00022 |
| Example 5 | 1:0.00051 |
| Example 6 | 1:0.0013 |
| Example 7 | 1:0.0026 |
| Example 8 | 1:0.0051 |
| Example 9 | 1:0.011 |

### Comparative Examples 2 and 3

The catalysts were prepared according to the substantially same method of Examples 1 and 2, except that the added amount of ammonium tungsten pentahydrate solution was different to achieve an atomic molar ratio of Mo to W as 1:0.025 (Comparative Example 2) and 1:0.03 (Comparative Example 3). The catalysts of Comparative Examples 2 to 3 were obtained to include the tungsten or tungsten oxide attached to the composite metal oxide, Mo_{1.0}V_{0.3}Te_{0.23}Nb_{0.12}Oₙ.

### Experimental Example

The direct oxidation reactions of propane using the catalysts of Examples 1 to 9 and Comparative Examples 1 to 3 were carried out as following methods.

That is, 0.1 g of each catalyst was charged into a fixed bed type reactor, and then reactant gas containing propane, oxygen, nitrogen and water was fed to the reactor with volumetric speed of 1,000 hr⁻¹ at 400° C. The molar ratio of propane:oxygen:nitrogen:water in the reactant gas was 8.8:14.8:39.3:37.6.

The propane contained in the reactant gas was converted into acrylic acid according to the gas-phase direct oxidation. When about 45% of propane was oxidized to other material, the conversion ratio of the propane to acrylic acid was measured.

In the direct oxidation reaction using each catalyst of Examples 1 to 9 and Comparative Examples 1 to 3, the conversion rates of acrylic acid were indicated in Table 2.

TABLE 2

| | acrylic acid conversion rate |
|---|---|
| Example No. | (45% propane oxidation) |
| Example 1 | 63.7 |
| Example 2 | 64.1 |
| Example 3 | 67.7 |
| Example 4 | 67.8 |
| Example 5 | 67.9 |
| Example 6 | 68.1 |
| Example 7 | 65.4 |
| Example 8 | 64.1 |
| Example 9 | 63.8 |
| Comparative Example 1 | 60.5 |
| Comparative Example 2 | 63.1 |
| Comparative Example 3 | 62.7 |

As referring to Table 2, the catalysts including tungsten or tungsten oxide attached to the surface of 4-membered composite metal oxide of molybdenum (Mo), vanadium (V), Tellurium (Te) and niobium (Nb) by non-chemical binding in Examples 1 to 9 showed the excellent catalytic activity and selectivity, compared to the catalyst of 4-membered composite metal oxide of molybdenum (Mo), vanadium (V), Tellurium (Te) and niobium (Nb) without including the attached tungsten or tungsten oxide in Comparative Example 1.

In addition, the catalyst of Examples 1 to 9 that satisfied the atomic molar ratio of W to Mo (0.00001:1 to 0.02:1), the yield of acrylic acid and selectivity was excellently improved, compared to the catalyst including the attached molar ratio of tungsten beyond the range in Comparative Examples 2 and 3. On the other hand, the catalysts in Comparative Examples 2 and 3 showed the acrylic acid conversion rate similar to that of the catalyst in Comparative Example 1, and thus did not improve the acrylic acid yield and selectivity.

The reasons are that the tungsten or tungsten oxide in the catalyst of Examples 1 to 9 acts independently as different catalytic site without inhibiting the catalytic activity and selectivity of the composite metal oxide alone, thereby improving the catalytic activity and selectivity.

Particularly, the catalysts of Examples 3 to 6 to satisfy the atomic molar ratio of W to Mo lower than about 0.002:1, improved the acrylic acid yield and selectivity even more.

1. A catalyst for gas-phase contact oxidation of a hydrocarbon, comprising a composite metal oxide of Molybdenum (Mo), Vanadium (V), Tellurium (Te) and Niobium (Nb); and a tungsten (W) or tungsten oxide attached to the composite metal oxide,
wherein an atomic molar ratio of the tungsten attached to the composite metal oxide to the molybdenum contained in the composite metal oxide ranges from 0.00001:1 to 0.02:1.

2. The catalyst for gas-phase contact oxidation of claim 1, wherein the composite metal oxide is represented by chemical formula I:
Mo_{1.0}VₐTe_{b}Nb_{c}Oₙ (I)
Where,
a, b, or c is independently an atomic molar ratio of Vanadium, Tellurium, or Niobium, provided that 0.01≦a≦1, 0.01≦b≦1, 0.01≦c≦1; and
n is an atomic molar ratio of Oxygen that is determined by valence and atomic molar ratio of Vanadium, Tellurium, and Niobium.

3. The catalyst for gas-phase contact oxidation of claim 1, wherein an atomic molar ratio of the tungsten attached to the composite metal oxide to the molybdenum contained in the composite metal oxide ranges from 0.0001:1 to 0.002:1.

4. The catalyst for gas-phase contact oxidation of claim 1, wherein the catalyst is used for the direct oxidation or ammoxidation of the hydrocarbon comprising propane or isobutane.

5. The catalyst for gas-phase contact oxidation of claim 4, wherein the catalyst is used for the direct oxidation or ammoxidation of the hydrocarbon comprising propane or isobutane to produce acrylic acid, methacrylic acid or acrylonitrile.

6. A method of preparing a catalyst for gas-phase contact oxidation of hydrocarbon according to claim 1, comprising the steps of:
preparing a first mixture of Molybdenum (Mo) precursor, Vanadium (V) precursor, Tellurium (Te) precursor, Niobium (Nb) precursor, and acid;
preparing a composite metal oxide of Molybdenum (Mo), Vanadium (V), Tellurium (Te) and Niobium (Nb) by calcining the first mixture;
preparing a second mixture of the composite metal oxide and tungsten precursor; and
calcining the second mixture.

7. The method of preparing a catalyst of claim 6, wherein the first mixture and the second mixture is an aqueous solution.

8. The method of preparing a catalyst of claim 6, wherein the acid is at least one selected from the group consisting of nitric acid, sulfuric acid, hydrochloric acid, carbonic acid, hypochlorous acid and fluoric acid.

9. A method of a gas-phase oxidation of hydrocarbon, comprising oxidizing the hydrocarbon in the presence of the catalyst according to claim 1 in gaseous phase.

10. The method of claim 9, wherein the hydrocarbon comprising propane or isobutane is oxidized by gas-phase direct oxidation or gas-phase ammoxidation in the presence of catalyst.

11. The method of claim 10, wherein the propane or isobutane is oxidized by gas-phase direct oxidation or gas-phase ammoxidation to selectively produce acrylic acid, methacrylic acid or acrylonitrile.

12. A method of a gas-phase oxidation of hydrocarbon, comprising oxidizing the hydrocarbon in the presence of the catalyst according to claim 2 in gaseous phase.

13. The method of claim 12, wherein the hydrocarbon comprising propane or isobutane is oxidized by gas-phase direct oxidation or gas-phase ammoxidation in the presence of catalyst.

14. The method of claim 13, wherein the propane or isobutane is oxidized by gas-phase direct oxidation or gas-phase ammoxidation to selectively produce acrylic acid, methacrylic acid or acrylonitrile.

15. A method of a gas-phase oxidation of hydrocarbon, comprising oxidizing the hydrocarbon in the presence of the catalyst according to claim 3 in gaseous phase.

16. The method of claim 15, wherein the hydrocarbon comprising propane or isobutane is oxidized by gas-phase direct oxidation or gas-phase ammoxidation in the presence of catalyst.

17. The method of claim 16, wherein the propane or isobutane is oxidized by gas-phase direct oxidation or gas-phase ammoxidation to selectively produce acrylic acid, methacrylic acid or acrylonitrile.
